(19) United States
(12) Patent Application Publication
(10) Pub. No.: US 2022/0064698 A1
(43) Pub. Date: Mar. 3, 2022

(54) MULTIPLEXED IMAGING REAGENT COMPOSITIONS AND KITS

(71) Applicant: Akoya Biosciences, Inc., Menlo Park, CA (US)

(72) Inventor: Peter J. Miller, Cambridge, MA (US)

(21) Appl. No.: 17/458,505

(22) Filed: Aug. 26, 2021

Related U.S. Application Data
(60) Provisional application No. 63/070,822, filed on Aug. 26, 2020.

(51) Int. Cl. C12Q 1/682; C12Q 1/6837

(52) U.S. Cl. CPC C12Q 1/682; C12Q 1/6837

(57) ABSTRACT

Methods for detecting multiple target analytes in a biological sample include: (a) contacting the biological sample with a plurality of different types of probes, where each different type of probe includes a capture moiety that selectively binds to a different target analyte in the sample, and an oligonucleotide having a sequence that is unique among other types of probes in the plurality of different types of probes; (b) binding an optical label to one of the different types of probes; (c) contacting the sample with a composition that includes at least one blocking agent, where the at least one blocking agent includes an oligonucleotide having a sequence that hybridizes to the oligonucleotide of another type of probe from among the different types of probes; and (d) obtaining an image of the sample that includes information corresponding to one or more locations of the one type of probe in the sample.

### CROSS-REFERENCE TO RELATED APPLICATIONS

This application claims priority to U.S. Provisional Patent Application No. 63/070,822, filed on Aug. 26, 2020, the entire contents of which are incorporated herein by reference.

### BACKGROUND

Immuno-labeling can be used to target molecules in samples such as cells, tissue, and other biological specimens. Attaining high sensitivity can enable reliable detection of low-abundance targets.

### SUMMARY

This disclosure features methods, reagent compositions, and kits for performing multiplexed imaging of tissue samples such as tissue sections. The reagent compositions and kits can be used to promote selective labeling of a subset of different types of oligonucleotide-coupled antibody from among a larger pool of different types of oligonucleotide-coupled antibodies in a biological sample. By ensuring that only one or a relatively small number (e.g., 2, 3, 4, 5, 6, 8, 10) of different types of oligonucleotide-coupled antibodies are labeled, the labeled antibodies can be imaged, identified, and quantitatively measured with high levels of accuracy, absent confounding effects arising from cross-labeling of multiple different antibody types. The reagent compositions and kits described herein can be used to perform highly multiplexed tissue imaging and marker quantitation via successive cycles of labeling, imaging, and label removal from the sample. The methods can also be used to selectively label relatively small numbers of different types of oligonucleotides, alone or coupled to moieties other than antibodies, from among a larger pool of such species, and are not restricted to only the labeling of antibody-coupled oligonucleotides.

In a first aspect, the disclosure features methods for detecting multiple target analytes in a biological sample, the methods including: (a) contacting a biological sample with a plurality of different types of probes, where each different type of probe includes a capture moiety that selectively binds to a different target analyte in the sample, and an oligonucleotide having a sequence that is unique among other types of probes in the plurality of different types of probes; (b) binding an optical label to one of the different types of probes, where the optical label includes an optical moiety linked to a labeling oligonucleotide, and where the labeling oligonucleotide includes a sequence that hybridizes to the oligonucleotide sequence of the one type of probe; (c) contacting the sample with a composition featuring at least one blocking agent, where the at least one blocking agent includes an oligonucleotide having a sequence that hybridizes to the oligonucleotide of another type of probe from among the different types of probes; and (d) obtaining an image of the sample that includes information corresponding to one or more locations of the one type of probe in the sample.

Embodiments of the methods can include any one or more of the following features.

The methods can include contacting the sample with the composition prior to binding the optical label to the one of the different types of probes. The methods can include contacting the sample with the composition during the binding the optical label to the one of the different types of probes. The methods can include contacting the sample with the composition after binding the optical label to the one of the different types of probes.

The capture moiety can include at least one of an antibody and an antibody fragment. The capture moiety can include an aptamer. The capture moiety can include at least one of a protein and a peptide. The capture moiety can include a ribonucleic acid.

Binding the optical label to the one of the different types of probes can include hybridizing the optical label to the one of the different types of probes.

The at least one blocking agent may not include an optical moiety or an enzyme. The optical moiety can include a fluorescent dye species. The optical moiety can include an enzyme.

The methods can include, prior to obtaining the image of the sample, exposing the sample to a second composition that includes a tyramide-conjugated optical moiety to deposit the optical moiety in the sample in proximity to the one of the different types of probes.

The composition can include multiple different types of blocking agents, and each different type of blocking agent can include an oligonucleotide having a sequence that hybridizes to the oligonucleotide of a different one of the other types of probes. The methods can include removing the optical label from the sample, and repeating steps (b) and (c) with at least one additional optical label.

The sample can include n different types of target analytes, and the composition can include (n−1) different types of blocking agents. The composition can include blocking agents featuring oligonucleotides with sequences that are complementary to oligonucleotides of one or more other types of probes from among the plurality of different types of probes.

The image can include fluorescence emission information for the sample. The plurality of different types of probes can include at least 20 different types of probes. The oligonucleotide of the probe can include at least 20 nucleotides.

A concentration of the optical label in the sample is M_{c}, and a concentration of a blocking agent in the sample can be 1.5 M_{c} or more. A value of a hybridization discrimination factor between two different types of probes and the optical label can be 100 or less.

Embodiments of the methods can also include any of the other features described herein, including combinations of features that are individually described in connection with different embodiments, in any combination unless expressly stated otherwise.

In another aspect, the disclosure features kits that include: a first composition featuring a plurality of different types of probes, where each different type of probe includes a capture moiety that selectively binds to a different target analyte, and an oligonucleotide having a sequence that is unique among other types of probes in the plurality of different types of probes; an optical label that includes an optical moiety linked to a labeling oligonucleotide, where the labeling oligonucleotide features a sequence that is at least partially complementary to the oligonucleotide sequence of one of the different types of detection molecules; and a second composition featuring at least one blocking agent, where the at least one blocking agent includes an oligonucleotide having a sequence that is at least partially complementary to the oligonucleotide sequence of another type of probe from among the different types of probes.

Embodiments of the kits can include any one or more of the following features.

The capture moiety can include at least one of an antibody, an antibody fragment, an aptamer, a protein, and a peptide. The capture moiety can include a ribonucleic acid. The at least one blocking agent may not include an optical moiety or an enzyme. The optical moiety can include a fluorescent dye species. The optical moiety can include an enzyme.

The first composition can include n different types of target analytes. The second composition can include a plurality of sub-compositions. Each sub-composition can include less than n different types of blocking agents. Each sub-composition can be housed within a separate container.

The second composition can include multiple different types of blocking agents, where each type of blocking agent includes an oligonucleotide having a sequence that is at least partially complementary to the oligonucleotide sequence of a different type of probe from among the different types of probes.

Embodiments of the kits can also include any of the other features described herein, including combinations of features that are individually described in connection with different embodiments, in any combination unless expressly stated otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the subject matter herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments are set forth in the accompanying drawings and the description below. Other features and advantages will be apparent from the description, drawings, and claims.

### DESCRIPTION OF DRAWINGS

FIG. 1A is a schematic diagram showing an example of a probe,

FIG. 1B is a schematic diagram showing an example of an optical label.

FIG. 2 is a schematic diagram showing an example of an optical label that selectively associates with one type of probe in a sample.

FIG. 3 is a flow chart showing a set of example steps for analysis of a biological sample.

FIG. 4 is a schematic diagram showing an example multispectral imaging system.

FIG. 5 is a schematic diagram showing an example controller.

Like reference symbols in the various drawings indicate like elements.

### DETAILED DESCRIPTION

### Introduction

A wide variety of different labeling and imaging techniques can be used for the detection of specific target analytes in biological samples. In some techniques, a capture moiety such as an antibody is conjugated to an oligonucleotide (e.g., a strand of DNA or RNA). Typically, a biological sample (e.g., a tissue section) is exposed to a plurality of different types of capture moieties, each of which is conjugated to a different type of oligonucleotide (e.g., where each different type of oligonucleotide has a different nucleotide sequence). Each type of capture moiety selectively binds to a specific type of target analyte (e.g., a biomarker) in the sample.

A specific type of target analyte in the sample can be selectively detected by associating an optical label (e.g., a fluorescent label) to the capture moiety that binds to the target analyte. To selectively interrogate specific target analytes, optical labels include an optical moiety (e.g., a fluorescent moiety) linked to an oligonucleotide sequence that is sufficiently complementary to the oligonucleotide linked to one of the capture moieties so that the oligonucleotides hybridizing, binding the optical label to the specific capture moiety at the location of the specific target analyte in the sample. The binding of the optical label to the specific capture moiety occurs nominally selectively to only that type of capture moiety, thereby selectively localizing in the sample only the target analyte is located. By subsequently measuring an optical signal from the optical label, the target analyte can be identified and quantified in the sample. After measuring the optical signal, the optical label can be de-hybridized and washed out of the sample. This process can be repeated many times, permitting multiplexed visualization of many different target analytes in a sample.

Technologies such as cyclic labeling, imaging, and de-labeling using CODEX™ reagents (available from Akoya Biosciences, Menlo Park, Calif.) have been developed to implement the methods described above. Additional aspects of the methods and reagents used in such multiplexed techniques are described for example in U.S. Pat. No. 10,370,698, the entire contents of which are incorporated herein by reference.

The methods and kits described herein can be used with a wide variety of different types of target analytes. Examples of such analytes include, but are not limited to, antigens, peptides, proteins, and other amino-acid containing moieties. Additional examples of such analytes include, but are not limited to, oligonucleotides, including oligonucleotides containing DNA bases, RNA bases, both DNA and RNA bases, and synthetic bases, nucleic acid fragments, and lipids.

The methods and kits described herein are suited for the identification and quantification of many different clinically relevant biomarkers in biological samples, particularly biomarkers that are expressed in tumor tissues, in the tumor microenvironment, and tissues representative of other disease states. Examples of such biomarkers that correspond to target analytes include, but are not limited to, tumor markers such as Sox10, S100, pan-cytokeratin, PAX5, PAX8; immune cell identifiers such as CD3, CD4, CD8, CD20, FoxP3, CD45RA, CD45LCA, CD68, CD163, CD11c, CD33, HLADR; activation markers such as Ki67, granzyme B; checkpoint-related markers such as TIM3, LAG3, PD1, PDL1, CTLA4, CD80, CD86, IDO-1, VISTA, CD47, CD26.

The methods and kits described herein can be used to analyze a variety of different types of biological samples. In some embodiments, the biological sample can be fresh, frozen, or fixed. The biological sample can be of animal origin, such as from a human, mouse, rat, cow, pig, sheep, monkey, rabbit, fruit fly, frog, nematode or woodchuck. The biological sample can include formalin-fixed paraffin-embedded (FFPE) tissue sections, frozen tissue sections, fresh tissue, cells obtained from a subject (e.g., via fine-needle aspirate or other technique), cultured cells, biological tissue, biological fluid, a homogenate, or an unknown biological sample.

In certain embodiments, the biological sample can be immobilized on a surface. For example, the surface can be a slide, a plate, a well, a tube, a membrane, or a film. In some embodiments, the biological sample can be mounted on a slide. In certain embodiments, the biological sample can be fixed using a fixative, such as an aldehyde, an alcohol, an oxidizing agent, a mercurial, a picrate, HOPE fixative, or another fixative. The biological sample may alternatively, or in addition, be fixed using heat fixation. Fixation can also be achieved via immersion or perfusion.

In some embodiments, the biological sample can be frozen. For example, the biological sample can be frozen at less than 0° C., less than −10° C., less than −20° C., less than −30° C., less than −40° C., less than −50° C., less than −60° C., less than −70° C., or less than −80° C.

In certain embodiments, the biological sample can be immobilized in a three dimensional form. The three dimensional form can include, for example, a frozen block, a paraffin block, or a frozen liquid. For example, the biological sample can be a block of frozen animal tissue in an optimal cutting temperature compound. The block of tissue can be frozen or fixed. In some embodiments, the block of tissue can be cut to reveal a surface which can be the surface contacted by first agent as discussed above.

In some embodiments, where the biological sample corresponds to a block, the block can be sliced to produce serial sections of the block, each of which can be analyzed according to the methods described herein. By doing so, three dimensional information (e.g., information as a function of depth within the sample) about the identity and/or quantity of one or more target analytes in the sample can be obtained.

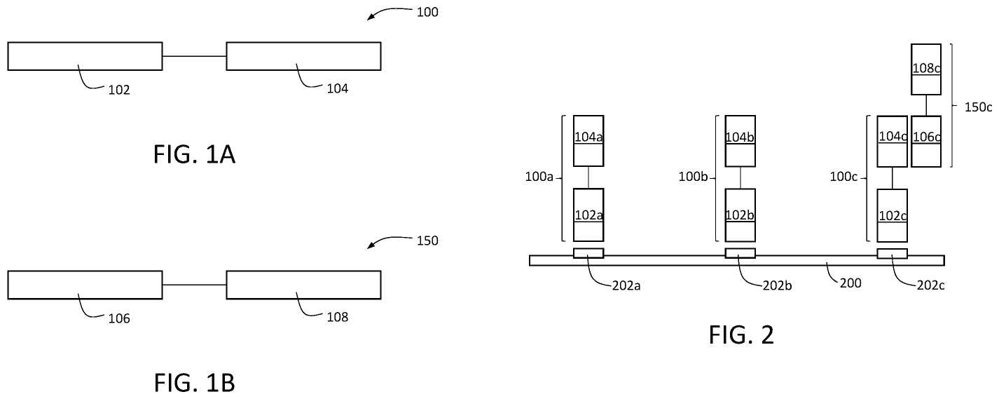

FIG. 1A is a schematic diagram showing an example of a probe 100 that specifically binds to a target analyte in a biological sample. Probe 100 includes a capture moiety 102 that is linked to an oligonucleotide 104. The selectivity of probe 100 arises from the specific interaction of capture moiety 102 with the target analyte.

In some embodiments, capture moiety 102 is an antibody (e.g., a primary antibody) or antibody fragment. The antibody or antibody fragment can include any one or more different types of antibody species, including but not limited to, an immunoglobulin G (IgG), an immunoglobulin M (IgM), a polyclonal antibody, a monoclonal antibody, a single-chain fragment variable (scFv) antibody, a nanobody, an antigen-binding fragment (Fab), and a diabody. Antibodies and antibody fragments can be of mouse, rat, rabbit, human, camelid, or goat origin. In some embodiments, the antibody or antibody fragment can be raised against a human, mouse, rat, cow, pig, sheep, monkey, rabbit, fruit fly, frog, nematode or woodchuck antigen. In certain embodiments, the antibody or antibody fragment can be raised against an animal, plant, bacteria, fungus, or protist antigen.

Capture moieties 102 that include an antibody or antibody fragment can selectively bind to target analytes including antigens, peptides, proteins, and other amino acid-containing species in the biological sample. Where capture moiety 102 is an antibody or antibody fragment and the target analyte is an antigen, binding occurs between the antigen epitope and the paratope of the antibody or antibody fragment. Where capture moiety 102 is an antibody or antibody fragment and the target analyte is a lipid, binding can occur between a recognition site on the antibody or antibody fragment and a head group of the lipid (e.g., a phospholipid head group).

In some embodiments, capture moiety 102 includes an oligonucleotide. The oligonucleotide can include DNA bases (e.g., A, C, G, T), RNA bases (e.g., A, C, G, U), and any combination of DNA and/or RNA bases. Capture moiety 102 can also include non-natural (e.g., synthetic) nucleotides, including DNA analogues and/or RNA analogues. Examples of such synthetic analogues include, but are not limited to, peptide nucleic acids, morpholino and locked nucleic acids, glycol nucleic acids, and threose nucleic acids.

Capture moieties 102 that include an oligonucleotide can selectively bind to a target analyte that includes nucleotide bases (e.g., a RNA, a DNA, or another species that includes one or more RNA bases and/or one or more DNA bases) via hybridization. In general, when capture moiety 102 includes an oligonucleotide, the length of the oligonucleotide can be any length, and is typically selected to ensure efficient and selective hybridization with the target analyte. In certain embodiments, for example, the oligonucleotide can include at least 5 (e.g., at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100) nucleotides.

In some embodiments, the oligonucleotide can have between 5-30, between 5-25, between 5-20, between 10-20, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides. In certain embodiments, the oligonucleotide can have no more than 5 (e.g., no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100) nucleotides.

In some embodiments, the oligonucleotide can be fully single stranded. Alternatively, in certain embodiments, the oligonucleotide can be partially double stranded. A partially double stranded region of the oligonucleotide can be at the 3′ end of the oligonucleotide, at the 5′ end of the oligonucleotide, or between the 5′ end and 3′ end of the oligonucleotide.

Probe 100 includes oligonucleotide 104. In general, oligonucleotide 104 includes multiple nucleotides. The nucleotides can include, for example, DNA bases (e.g., A, C, G, T), RNA bases (e.g., A, C, G, U), and any combination for DNA and/or RNA bases. Oligonucleotide 104 can also include non-natural (e.g., synthetic) nucleotides, including DNA analogues and/or RNA analogues. Examples of such synthetic analogues include, but are not limited to, peptide nucleic acids, morpholino and locked nucleic acids, glycol nucleic acids, and threose nucleic acids.

The sequence of bases in oligonucleotide 104 can generally be any sequence. Moreover, in general, nucleotides and other moieties in oligonucleotide 104 can be conjugated via natural and/or non-natural (e.g., synthetic) linkages.

In some embodiments, oligonucleotide 104 includes one or more nucleotides that are capable of base pairing with high reliability with a complementary nucleotide. Examples of such nucleotides include, but are not limited to, 7-deaza-adenine, 7-deaza-guanine, adenine, guanine, cytosine, thymine, uracil, 2-deaza-2-thio-guanosine, 2-thio-7-deaza-guanosine, 2-thio-adenine, 2-thio-7-deaza-adenine, isoguanine, 7-deaza-guanine, 5,6-dihydrouridine, 5,6-dihydrothymine, xanthine, 7-deaza-xanthine, hypoxanthine, 7-deaza-xanthine, 2,6 diamino-7-deaza purine, 5-methyl-cytosine, 5-propynyl-uridine, 5-propynyl-cytidine, 2-thio-thymine, and 2-thio-uridine.

In certain embodiments, oligonucleotide 104 can correspond to, or contain one or more fragments of, specialized nucleic acid species. For example, oligonucleotide 104 can correspond to, or contain one or more fragments of, a locked nucleic acid (LNA), a peptide nucleic acid (PNA), an unlocked nucleic acid (UNA), and/or a morpholino oligomer.

The length of oligonucleotide 104 (e.g., the number of nucleotides in oligonucleotide 104) can generally be selected as desired to ensure efficient and selective hybridization interactions. In some embodiments, oligonucleotide 104 can include at least 5 (e.g., at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100) nucleotides.

In some embodiments, oligonucleotide 104 can have between 5-30, between 5-25, between 5-20, between 10-20, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides.

In certain embodiments, oligonucleotide 104 can have no more than 5 (e.g., no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100) nucleotides.

In some embodiments, oligonucleotide 104 can be fully single stranded. Alternatively, in certain embodiments, oligonucleotide 104 can be at least partially double stranded. A partially double stranded region of oligonucleotide 104 can be at the 3′ end of the oligonucleotide, at the 5′ end of the oligonucleotide, or between the 5′ end and 3′ end of the oligonucleotide.

As shown in FIG. 1A, capture moiety 102 is linked to oligonucleotide 104 in probe 100. The linkage between capture moiety 102 and oligonucleotide 104 can be implemented in various ways. In some embodiments, capture moiety 102 and oligonucleotide 104 can be linked directly via a covalent or non-covalent bond. That is, capture moiety 102 and oligonucleotide 104 can be linked directly via a bond, with no intervening moiety or structure between capture moiety 102 and oligonucleotide 104.

In certain embodiments, capture moiety 102 and oligonucleotide 104 can be linked via a primary-secondary antibody pair. To label a target analyte, the target analyte is exposed to a first labeling agent that includes capture moiety 102 which is, or is conjugated to, a primary antibody. Once the first labeling agent selectively binds to the target analyte, a second labeling agent is introduced that includes oligonucleotide 104 conjugated to a secondary antibody. The secondary antibody selectively binds to the primary antibody, linking the capture moiety 102 (which may be the primary antibody or another moiety) and oligonucleotide 104, and yielding a construct in which the target analyte is bound to the capture moiety 102 which is in turn linked to oligonucleotide 104.

In some embodiments, the linkage between capture moiety 102 and detection moiety 104 can be implemented as a double-stranded nucleic acid (e.g., hybridized nucleic acid strands that are at least partially complementary). For example, the target analyte can be exposed to a first labeling agent that includes capture moiety 102 linked to a first nucleic acid, which functions as a part of the linkage between capture moiety 102 and oligonucleotide 104. Once the first labeling agent selectively binds to the target analyte, a second labeling agent is introduced that includes oligonucleotide 104 linked to a second nucleic acid that functions as another part of the linkage. The second nucleic acid is at least partially complementary to the first nucleic acid, and selectively hybridizes to the first nucleic acid, so that the target analyte is bound to a construct that includes capture moiety 102 linked to oligonucleotide 104.

In some embodiments, the first nucleic acid can be a nucleic acid sequence that is contiguous with capture moiety 102. In other words, capture moiety 102 and the first nucleic acid can form a continuous nucleic acid sequence in which a portion of the nucleic acid sequence functions as capture moiety 102 (i.e., a capture region), and a portion of the nucleic acid sequence functions as the first nucleic acid (i.e., a linking nucleic acid sequence). The continuous nucleic acid sequence can be single-stranded or double-stranded.

In certain embodiments, the second nucleic acid can be a nucleic acid sequence that is contiguous with oligonucleotide 104. That is, the second nucleic acid and oligonucleotide 104 can form a continuous nucleic acid sequence in which a portion of the nucleic acid sequence functions as the second nucleic acid (i.e., a linking nucleic acid region), and a portion of the nucleic acid sequence functions as oligonucleotide 104. The continuous nucleic acid sequence can be single-stranded or double-stranded.

In some embodiments, capture moiety 102 can be linked to the first nucleic acid through conjugation, e.g., capture moiety 102 can be covalently bonded to the first nucleic acid. Any of a wide variety of different linkages can be used to covalently bond capture moiety 102 and the first nucleic acid, as discussed below. In certain embodiments, oligonucleotide 104 can be linked to the second nucleic acid through covalent bonding, using any of the different linkages described herein.

In some embodiments, the first and second nucleic acids that function as portions of the linkage between capture moiety 102 and oligonucleotide 104 do not directly hybridize. Instead, the first and second nucleic acids each hybridize to a portion of a bridging oligonucleotide that includes nucleic acid sequences that are at least partially complementary to each of the first and second nucleic acids. Bridging oligonucleotides can be single-stranded, such that a portion of the bridging oligonucleotide hybridizes to the first nucleic acid and a portion of the bridging oligonucleotide hybridizes to the second nucleic acid. Bridging oligonucleotides can be partially double-stranded, with overhangs on one or both strands that hybridize to the first and second nucleic acids to form the linkage between capture moiety 102 and oligonucleotide 104.

Bridging oligonucleotides can be linear such that a single capture moiety is linked to a single reporter moiety. Alternatively, bridging oligonucleotides can be branched, and can include a single nucleic acid sequence that hybridizes to capture moiety 102, and multiple nucleic acid sequences that hybridize to oligonucleotide 104. As a result, a single capture moiety 102 can be linked to multiple oligonucleotides 104, allowing for amplification of optical signals that correspond to the target analyte to which capture moiety 102 selectively binds.

In certain embodiments, the linkage between capture moiety 102 and oligonucleotide 104 can be implemented as any of a variety of aliphatic and/or aromatic linking species. Further, as discussed above, in some embodiments, capture moiety 102 can be covalently bonded to the first nucleic acid, either via a direct covalent bond, or via any of a variety of aliphatic and/or aromatic linking species. Further still, as discussed above, in certain embodiments, oligonucleotide 104 can be covalently bonded to the second nucleic acid, either via a direct covalent bond, or via any of a variety of aliphatic and/or aromatic linking species.

Examples of linking species that can be used to directly link capture moiety 102 and oligonucleotide 104, to link capture moiety 102 and the first nucleic acid, and/or to link oligonucleotide 104 and the second nucleic acid, include—but are not limited to—C₁₋₂₀ cyclic and non-cyclic alkyl species, C₂₋₂₀ cyclic and non-cyclic alkene species, C₂₋₂₀ cyclic and non-cyclic alkyne species, and C₃₋₂₄ aromatic species. Any of the foregoing species can include heteroatoms such as, but not limited to, O, S, N, and P. Any of the foregoing species can also include one or more substituents selected from the group consisting of: halide groups; nitro groups; azide groups; hydroxyl groups; primary, secondary, and tertiary amine groups; aldehyde groups; ketone groups; amide groups; ether groups; ester groups; thiocyanate groups; and isothiocyanate groups.

FIG. 1B is a schematic diagram showing an example of an optical label 150. Optical label 150 includes an oligonucleotide 106 linked to an optical moiety 108. In general, the structure (i.e., the nucleotide sequence) of oligonucleotide 106 is sufficiently complementary to oligonucleotide 104 such that, when oligonucleotide 106 contacts oligonucleotide 104, the two oligonucleotides hybridize, binding optical label 150 to probe 100. Nominally, the sequence of oligonucleotide 106 is also sufficiently different from other types of probes that may be present in the biological sample so that oligonucleotide 106 does not hybridize with those other types of probes. As a result, optical label 150 is localized in the sample at only locations where the target analyte is present.

In general, oligonucleotide 106 can include any of the features described above for oligonucleotide 104. Oligonucleotide 106 can, in some embodiments, include the same number of nucleotides as oligonucleotide 104. Alternatively, in certain embodiments, oligonucleotide 106 can include a different number of nucleotides.

Oligonucleotide 106 can have the same or different strand structure as oligonucleotide 104. That is, oligonucleotide 106 can be single stranded, double stranded, or partially double stranded, irrespective of the structure of oligonucleotide 104. Oligonucleotide 106 can generally include any number of double stranded regions, as described above for oligonucleotide 104, extending over a portion of the total length of oligonucleotide 106.

As discussed above, oligonucleotide 106 hybridizes to oligonucleotide 104 via base pairing so that probe 100 and optical label 150 are co-localized in the sample at the location of the target analyte. The efficiency of hybridization is related in part to the extent of complementarity between the sequences of the oligonucleotides. As used herein, the percentage to which the sequences of the two sequences are complementary refers to the percentage of nucleotides in the shorter of the two sequences that have a complementary counterpart at a complementary location in the other sequence, such that the two counterparts pair during hybridization. In some embodiments, for example, the sequences of the two oligonucleotides are at least 70% (e.g., at least 75%, at least 80%, at least 85%, at least 90%, at least 95%, at least 99%) complementary.

As used herein, the term “at least partially complementary” means that two nucleotide sequences are sufficiently complementary that they hybridize. In general, two nucleotide sequences are at least partially complementary if their sequences are at least 50% complementary.

In general, oligonucleotide 106 includes at least one binding region that hybridizes to a corresponding binding region of oligonucleotide 104. The binding region can be located at the 3′ end, at the 5′ end, or intermediate between the two ends, of oligonucleotide 106. Where oligonucleotide 106 includes multiple binding regions, any of the binding regions can be located as above.

In some embodiments, the binding region of oligonucleotide 106 is at least partially complementary to, and hybridizes with, the 3′ end of oligonucleotide 104. In certain embodiments, the binding region of oligonucleotide 106 is at least partially complementary to, and hybridizes with, the 5′ end of oligonucleotide 104.

In certain embodiments, the binding region of oligonucleotide 106 is at least partially complementary to, and hybridizes with, an intermediate region of oligonucleotide 104. In some embodiments, the binding region of oligonucleotide 106 is at least partially complementary to, and hybridizes with, the entire oligonucleotide 104. In certain embodiments, the binding region of oligonucleotide 104 is at least partially complementary to, and hybridizes with, the entire oligonucleotide 106.

In certain embodiments, one or both of oligonucleotides 104 and 106 includes multiple binding regions separated by one or more non-binding regions. In general, each of the binding regions can have any of the properties discussed above in connection with oligonucleotides 104 and 106 and their respective binding regions.

Non-binding regions in oligonucleotides 104 and 106 can be formed by and/or include a variety of different linking species, including non-complementary nucleotide sequences and spacer moieties that do not include nucleotides. Non-binding regions can have the same or different geometric lengths, and binding regions can have the same or different lengths (e.g., the same or different numbers of nucleotides). Within each oligonucleotide (e.g., 104 and/or 106), binding regions and non-binding regions can have the same or different lengths.

In some embodiments, capture moiety 102 can be conjugated to multiple oligonucleotides 104 in probe 100. In general, each of the oligonucleotides 104 has the same nucleotide sequence, so that the oligonucleotide 106 can hybridize with any of the oligonucleotides 104. In general, 2 or more (e.g., 3 or more, 4 or more, 5 or more, 6 or more, 7 or more, 8 or more, or even more) oligonucleotides 104 can be conjugated to capture moiety 102. By conjugating more than one oligonucleotide 104 to capture moiety 102, additional optical labels can be selectively deposited in the sample at the location of the target analyte, thereby enhancing the measurement of detection signals from the sample that correspond to the target analyte.

Optical label 150 includes one or more optical moieties 108. In FIG. 1B, optical label 150 includes a single optical moiety 108 linked to oligonucleotide 106 for purposes of discussion. More generally, however, optical label 150 can include 1 or more (e.g., 2 or more, 3 or more, 4 or more, 5 or more, 6 or more, 8 or more, 10 or more, or even more) optical moieties 108 linked to oligonucleotide 106.

A variety of different optical moieties 108 can be used, depending upon the nature of the methodology used to identify and quantify target analytes in the sample. In some embodiments, for example, optical moiety 108 includes a dye. As used herein, a “dye” is a moiety that interacts with incident light, and from which emitted light can be measured and used to detect the presence of the dye in a sample. In general, a dye can be a fluorescent moiety, an absorptive moiety (e.g., a chromogenic moiety), or another type of moiety that emits light, and/or modifies incident light passing through or reflected from a sample where the dye is present so that the presence of the dye can be determined by measuring changes in transmitted or reflected light from the sample.

In certain embodiments, the optical moiety can include a hapten. The hapten can subsequently (or concurrently) be bound to a dye moiety to provide an optical moiety that can be detected by measuring emitted, transmitted, or reflected light from the sample.

When the optical moiety of optical label 150 includes a dye, a wide variety of different dyes can be used. For example, the dye can be a xanthene-based dye, such as a fluorescein dye and/or a rhodamine dye. Examples of suitable fluorescein and rhodamine dyes include, but are not limited to, fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110.

The dye can also be a cyanine-based dye. Suitable examples of such dyes include, but are not limited to, the dyes Cy3, Cy5 and Cy7. The dye can also be a coumarin dye (e.g., umbelliferone), a benzimide dye (e.g., any of the Hoechst dyes such as Hoechst 33258), a phenanthridine dye (e.g., Texas Red), an ethidium dyes, an acridine dyes, a carbazole dye, a phenoxazine dye, a porphyrin dye, a polymethine dye (e.g., any of the BODIPY dyes), and a quinoline dye.

When the dye is a fluorescent moiety, the dye can be a moiety corresponding to any of the following non-limiting examples and/or derivatives thereof: pyrenes, coumarins, diethylaminocoumarins, FAM, fluorescein chlorotriazinyl, fluorescein, Rl 10, JOE, R6G, tetramethylrhodamine, TAMRA, lissamine, napthofluorescein, Texas Red, Cy3, and Cy5.

In certain embodiments, the dye can include one or more quantum dot-based species. Quantum dot-based fluorophores are available with fluorescence emission spectra in many different spectral bands, and suitable quantum dot-based dyes can be used as labeling species in the methods described herein.

As shown in FIG. 1B, oligonucleotide 106 and optical moiety 108 are linked in optical label 150. In general, the linkage between oligonucleotide 106 and optical moiety 108 can correspond to any of the linkages discussed above in connection with probe 100.

Biological samples typically include multiple analytes of interest, and the methods and kits described herein can be used to perform multiplexed labeling and detection of target analytes. FIG. 2 is a schematic diagram of a sample 200 that includes three different types of target analytes 202a-220c. The different types of target analytes can each independently be any of the different types of target analytes described herein. In some embodiments, for example, the different types of target analytes can be different proteins, antigens, peptides, or other amino acid-containing species. In certain embodiments, the different types of target analytes can be different types of nucleic acids (e.g., RNAs). In some embodiments, the different types of target analytes can include combinations of any of the different types of target analytes described herein (e.g., proteins, antigens, peptides, amino acid-containing species, and nucleic acids such as RNAs).

To detect and optionally quantify each of the different types of target analytes in sample 200, the sample is exposed to probes 100a-100c that selectively bind, respectively, to target analytes 202a-202c in the sample. Probes 100a-100c include capture moieties 102a-102c and oligonucleotides 104a-104c, respectively. Probes 100a-100c can each independently have any of the properties discussed herein in connection with probe 100. Each of probes 100a-100c selectively binds to only one type of target analyte 202a-202c, so that each type of target analyte in sample 200 is bound to a different type of probe.

An important aspect of probes 100a-100c is that in general, the nucleotide sequences of oligonucleotides 104a-104c of the probes differ. This allows each of the probes 100a-100c to be selectively associated with a different optical label for detection. As such, different optical labels can be localized in the sample at locations corresponding to the different target analytes 202a-202c, allowing each of the target analytes to be separately identified and quantified.

To detect target analyte 202c for example, the sample is contacted with optical label 150c, which includes oligonucleotide 106c and optical moiety 108c. As shown in FIG. 2, oligonucleotide 106c is complementary to oligonucleotide 104c of probe 100c, so that oligonucleotide 106c selectively hybridizes to oligonucleotide 104c, but not to oligonucleotide 104a or 104b. As a result, optical label 150c associates/binds selectively with probe 100c in sample 200, and is spatially localized in sample 200 only where target analyte 202c is located.

Following binding of optical label 150c to probe 100c, an optical signal arising from optical label 150c is measured (e.g., a fluorescence emission signal). Measurement of such signals can be performed, for example, by obtaining an image of sample 200. The measured optical signal indicates the presence of optical label 150c—and therefore target analyte 202c—at specific locations within the sample, allowing for spatially resolved identification of the target analyte. Further, by measuring the intensity of the optical signal at different locations within the sample (e.g., the spatially-resolved fluorescence emission intensity at specific pixel locations within an image of the sample), the amount of the target analyte at specific locations in the sample can be quantified.

After the optical signal arising from optical label 150c has been measured, target analytes 202a and/or 202b can also be identified and/or quantified in sample 200. To identify these target analytes, optical label 150c is typically (but optionally) first removed from sample 200 by dehybridization or another method, or inactivated in sample 200. Dehybridization can be accomplished using various methods including, but not limited to: exposure to one or more chaotropic reagents; thermally-induced dehybridization via heating; toehold mediated strand displacement (TMSD); and enzymatic strand displacement using enzymes such as RNAse, DNAse.

In certain embodiments, optical labels (or portions of optical labels) can be removed from sample 200 using one or more reducing agents that cleave covalent bonds that link an optical moiety to an oligonucleotide in an optical label. The cleaved optical moieties can then be washed from the sample. The oligonucleotides can optionally remain hybridized to probes in the sample. A variety of different reducing agents can be used for this purpose. For example, tri(2-carboxyethyl)phosphine (TCEP) can be used to cleave optical moieties that are linked via disulfide bonds to oligonucleotides in optical labels.

In some embodiments, optical labels are not removed from the sample, but are instead inactivated so that they do not generate optical signals in subsequent detection cycles. Various methods can be used for inactivation of optical labels. For example, in certain embodiments, chemical bleaching can be used to inactivate optical labels.

After optional removal of label 150c, sample 200 is contact with optical labels that selectively associate with probes 100a and 100b, respectively, in the manner discussed above, to selectively localize these optical labels in the sample at locations corresponding to target analytes 202a and 202b, respectively. Optical signals measured from the sample that correspond to the localized optical labels can then be used to identify and/or quantify target analytes 202a and 202b in a spatially-resolved manner within sample 200.

In the foregoing example, when the sample contains multiple target analytes of interest, the target analytes (e.g., target analytes 202a-202c) are first contacted with a set of different types of probes (e.g., probes 100a-100c), where each type of probe selectively binds to one of the different types of target analytes. Then, the different types of probes are individually associated with a corresponding optical label, and a measurement signal for the corresponding type of target analyte bound to the type of probe is detected, by sequentially exposing the sample to different types of optical labels. For a sample that contains multiple target analytes of interest, the different types of target analytes are detected in successive cycles of analysis, with each cycle involving associating a single type of probe in the sample with an optical label, measuring a signal arising from the optical label, and optionally removing or inactivating the optical label. To detect N different types of target analytes, N cycles of analysis are performed.

More generally, however, one or more analysis cycles can involve contacting the sample with more than one different type of optical label. In such circumstances, the different types of optical labels selectively associate with different types of counterpart probes, and multiple optical signals corresponding to the different types of optical labels are measured within each cycle. The multiple optical signals can be used to identify and/or quantify multiple different types of target analytes in a spatially-localized manner within the sample in each cycle.

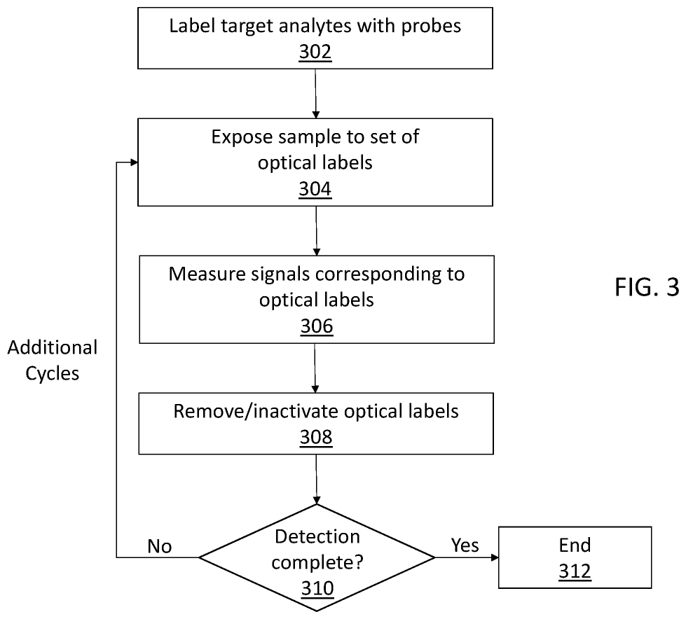

FIG. 3 is a flow chart that shows a set of example steps for detecting and quantifying multiple types of target analytes in a sample. In step 302, the multiple types of target analytes are labeled with different types of probes 100 as described above. Each type of probe selectively binds to a different type of target analyte. Furthermore, as discussed above, each type of probe includes a different type of oligonucleotide 104.

Next, in step 304, the sample is exposed to a set of one or more optical labels 150. The set of optical labels typically includes between 1 and 8 different optical labels (e.g., two, three, or four different optical labels), but can generally include any number of optical labels in each cycle of the flow chart of FIG. 3. Each type of optical label includes a different type of oligonucleotide 106. For a particular type of optical label, if oligonucleotide 106 of the optical label is complementary to oligonucleotide 104 of one of the types of probes in the sample, the oligonucleotides hybridize, associating the optical label with the probe, such that the optical label is localized in the sample at locations where the target analyte to which the type of probe is bound is located. As such, different target analytes within each cycle of the flow chart can be labeled with different optical moieties, and can be identified based on measured optical signals that correspond to the different optical moieties.

To increase the efficiency with which different types of target analytes are identified (e.g., by reducing the number of detection cycles), the set of optical labels can be selected such that, for at least one (and generally, more than one) cycle, multiple different optical labels of the introduced set each selectively associate with one of the different probe types, and generate optical signals. In this manner, multiple types of target analytes can be identified in a single detection cycle, reducing the number of cycles required to fully elucidate all of the target analytes present in the sample. By selecting the optical label set in each cycle such that each of multiple different optical labels selectively associates with one of the different types of probes in the sample, the number of detection cycles can be more efficiently utilized to identify the different types of probes, and therefore, the different target analytes in the sample.

Next, in step 306, optical signals corresponding to the optical labels are measured. In some embodiments, the optical signals are measured by obtaining one or more images (e.g., multispectral images) of the sample. To obtain the one or more images, the sample is exposed to incident light, and signal radiation generated by the optical labels (e.g., fluorescence emission) is detected using an imaging detector such as a CCD array or CMOS-based array detector.

In general, each of the different optical labels in the sample generates signal radiation according to a different spectral distribution, and is therefore associated with a different detection channel. In practice, signal radiation in different detection channels can be detected in a variety of ways. In some embodiments, where each detection channel is well separated spectrally from the other detection channels, the signal radiation generated by each different type of labeling agent or optical label is relatively well isolated spectrally in a distinct detection channel. As such, signal radiation attributable to each of the different types of labeling agents or optical labels can readily be isolated and detected by spectral filtering (e.g., with a plurality of optical bandpass filters) and/or by using a spectrally resolving detector, such as a grating, prism, or other spectrally dispersive element in conjunction with a CCD array or CMOS-based array detector.

In certain embodiments, the spectral distributions of signal radiation generated by the different optical labels may overlap to a degree that is not insignificant, such that optical filtering and spectral dispersion methods alone are insufficient to isolate signal radiation generated by each of the different labeling agents or optical labels. Because the spectral distributions of the signal radiation are spectrally convolved to some extent, accurate detection of signals generated by each of the optical labels may therefore involve more complex spectral deconvolution techniques to accurately separate and assign measured signals to specific labeling agents or optical labels.

In such circumstances, sample images that include signal radiation from multiple different optical labels can optionally be decomposed into a set of images, in which each image in the set corresponds substantially only to signal radiation from one optical moiety. A variety of methods can be used to perform such decompositions, including for example spectral unmixing methods that involve performing an eigenvector decomposition of the measured optical signals into individual contributions from “pure” spectral components (e.g., contributions from each optical label). Methods for spectral unmixing are described, for example, in U.S. Pat. Nos. 10,126,242 and 7,555,155, and in PCT Patent Publication No. WO2005/040769, the entire contents of each of which are incorporated herein by reference.

Step 306 yields a set of one or more images of the sample. Particular pixels at a common location in the set of images correspond to the same location in the sample, which is represented by the common pixel location in the images. Collectively, pixels across the set of images that correspond to a common pixel location are associated with optical signals generated by optical labels at the corresponding location in the sample. Because the optical signals generated by each different type of optical label in a detection cycle are known, the presence or absence of each type of target analyte in the sample at each pixel location can be determined. Further, the measured intensities of optical signals corresponding to the different types of target analytes at each pixel can be used to quantify the amount of each type of target analyte in a spatially-resolved manner within the sample.

Following step 306, the set of optical labels can be inactivated or removed from the sample in step 308. A variety of different methods can be used in step 308 for removal or inactivation of optical labels, as described above.

Next, in step 310, if analysis of the target analytes present in the sample is complete, then the workflow ends. However, if analysis is not complete, one or more additional cycles of steps 304, 306, and 308 are performed. In each additional cycle, a set of optical labels is optionally introduced into the sample, optical signals corresponding to the optical labels are measured and optionally decomposed as described above, and the optical labels can optionally be inactivated or removed from the sample. The workflow shown in FIG. 3 can be repeated for any number of cycles to detect and quantify target analytes in the sample.

Additional aspects of the methods for labeling and identifying target analytes in biological samples are described in PCT Patent Publication No. WO 2020/163397, in U.S. Provisional Patent Application No. 63/229,064, and in U.S. Pat. No. 10,370,698, the entire contents of each of which are incorporated by reference herein.

Reduction of Hybridization Cross Reactivity in Multiplexed Analytical Methods

As discussed above, an important aspect of the methods for labeling target analytes with optical labels described herein is the selective association (via hybridization) of optical labels with probes. Different types of probes 100 include different types of oligonucleotides 104. Similarly, different types of optical labels 150 include different types of oligonucleotides 106. When oligonucleotide 106 of one type of optical label 150 is complementary to oligonucleotide 104 of one type of probe, the optical label 150 associates (e.g., binds) to the probe via hybridization, localizing the optical label 150 in the sample at locations where the probe's corresponding target analyte is located.

When the foregoing specificity between optical labels and probes is maintained, measured optical signals arising from different optical labels can therefore be unambiguously attributed to specific target analytes in the sample. Perfect specificity implies that cross-hybridization among oligonucleotides does not occur. In other words, each of different types of oligonucleotides 106 in each of the different types of optical labels 150 is complementary to, and hybridizes only to, one of the different types of oligonucleotides 104 among the different types of probes 100, ensuring that different target analytes are selectively and reproducibly labeled for detection.

In practice, however, hybridization cross-reactivity (also referred to herein as “cross hybridization”)—in which an oligonucleotide 106 of a particular optical label exhibits some degree of hybridization non-specificity by hybridizing to more than one different oligonucleotide 104 of different probes 100—can affect the selectivity of the methods. In general, when measured optical signals arising from particular optical labels cannot be unambiguously attributed to specific counterpart probes (and the target analytes to which they bind), identifying and spatially localizing target analytes may be more difficult and prone to error. Further, when an optical label that nominally selectively associates (e.g., via hybridization) to a probe that binds to a weakly expressed target analyte also hybridizes to some extent with a probe that binds to another more abundant target analyte, measured fluorescence emission from the optical label associated with the more abundant target analyte can contribute spurious measured optical signal, which is erroneously attributed to the weakly expressed molecular target. This can make weakly expressed target analytes considerably more difficult to unambiguously detect than abundant target analytes in the sample.

The methods, compositions, and kits in this disclosure can be used to reduce the effects of optical label cross-hybridization, thereby improving the specificity and sensitivity with which weakly expressed targets can be reliably detected, and reducing false detection events.

By way of illustration, consider target analyte A, which is selectively bound by probe Da which in turn contains oligonucleotide 104 sequence Sₐ. Target analyte A is present in a sample containing other, possibly more abundant, target analytes {B, C, D . . . }, each of which is selectively bound to corresponding probes {D_{b}, D_{c}, D_{d} . . . }. Each of the corresponding probes contains an oligonucleotide 104 {S_{b}, S_{c}, S_{d} . . . } which is unique relative to the other oligonucleotides 104, and functions as a barcode sequence for its respective probe and target analyte. An optical label Rₐ for target analyte A includes an oligonucleotide 106 having a sequence (e.g., a barcode sequence) S′ₐ which is a countersense sequence to barcode sequence Sₐ. Oligonucleotide 106 with barcode sequence S′ₐ is linked to an optical moiety 108 within the optical label Rₐ such as a fluorescent dye or another type of dye, to an enzyme such as horseradish peroxidase (HRP), or to another moiety that can be used to generate a signal that can be measured. Measured optical signals from an optical moiety can be directly imaged to visualize target analyte A in the sample.

The subsequent discussion focuses on examples of methods in which optical label Rₐ (e.g., optical label 150) includes an oligonucleotide 106 linked to an optical moiety 108. However, it should be understood that the methods, reagents, and kits described herein can also be used when optical label 150 includes an oligonucleotide 106 linked to an enzyme such as HRP (i.e., in the example above, HRP or another enzyme is linked to sequence S′ₐ rather than an optical moiety). The enzyme can be used to catalyze deposition of TSA-conjugated optical moieties such as dye molecules at the location of sequence S′ₐ in the sample, facilitating amplification of measured optical signals that correspond to target analyte A. The steps, reagents, and kits described herein to reduce hybridization cross-reactivity are equally applicable to such optical labels 150, without limitation. Additional aspects of TSA-based labeling methods are described in PCT Patent Publication No. WO 2020/163397 and in U.S. Patent Application Publication No. 2021/0222234, the entire contents of each of which are incorporated by reference herein.

Unless perfect stringency occurs between sequences Sₐ and S′ₐ, there will be some degree of hybridization cross-reactivity between sequence S′ₐ and the sequences of oligonucleotides 104 of probes {D_{b}, D_{c}, D_{d} . . . }. Consequently, the barcode sequence S′ₐ intended for Sₐ has some probability of hybridizing with one or more of {S_{b}, S_{c}, S_{d} . . . }. This can be understood as a false-positive problem, and it results in a readout signal for target species {B, C, D . . . }. The likelihood of hybridizing with sequences {S_{b}, S_{c}, S_{d} . . . } depends on the relative abundances of these sequences, and the inherent hybridization cross-reactivity between sequence S′ₐ and {S_{b}, S_{c}, S_{d} . . . }.

In many circumstances, it is important to be able to detect target analyte A even when A is only expressed very weakly in the sample. Hybridization cross-reactivity interferes with such detection as noted above. Further, when methods are used to amplify measurement signals corresponding to certain target analytes (e.g., by using HRP-catalyzed deposition of TSA-linked optical moieties, as described above), spurious signals arising from hybridization cross-reactivity are also amplified, further complicating detection. Further still, for applications in which quantitative measurement of optical signals is important—for example, to quantify the amount of one or more target analytes present in the sample, in a spatially-resolved manner—hybridization cross-reactivity introduces spurious signals that distort quantitative spatial expression profiles for target analytes.

Accordingly, in certain embodiments, the sample can be contacted with sequence-specific blocking agents when optical label Rₐ is introduced into the sample. The blocking agents typically consist of oligonucleotides with sequences {S′_{b}, S′_{c}, S′_{d} . . . }. The oligonucleotides are similar to oligonucleotides 106 of optical labels 150, and can generally have any of the properties of oligonucleotides 106 discussed herein.

However, the oligonucleotides of the blocking agents are not coupled to an optical moiety 108 (e.g., a fluorescent dye) or to an enzyme such as HRP. The oligonucleotide blocking agents with sequences {S′_{b}, S′_{c}, S′_{d} . . . } preferentially hybridize to barcode sequences {S_{b}, S_{c}, S_{d} . . . }, which are associated with probes {D_{b}, D_{c}, D_{d} . . . } that bind to target species {B, C, D . . . }. Accordingly, the likelihood that barcode sequence S′ₐ will hybridize to a barcode sequence other than Sₐ is markedly reduced or effectively eliminated, as barcode sequences {S_{b}, S_{c}, S_{d} . . . } of probes {D_{b}, D_{c}, D_{d} . . . } are not available for hybridization by barcode sequence S′ₐ. In effect, because the stringency of hybridization between respective blocking agent sequences {S′_{b}, S′_{c}, S′_{d} . . . } and barcode sequences {S_{b}, S_{c}, S_{d} . . . } of probes {D_{b}, D_{c}, D_{d} . . . } is greater than the stringency of hybridization of sequence S′ₐ to barcode sequences {S_{b}, S_{c}, S_{d} . . . }, hybridization cross-reactivity is substantially reduced.

In general, blocking agents can be introduced at various stages of an analysis protocol. Referring for example to FIG. 3, in some embodiments, blocking agents can be introduced prior to the exposure of the sample to optical labels in step 304. In this manner, the barcode sequences of oligonucleotides 104 of probes 100 are already “blocked” when optical labels 150 are introduced.

In certain embodiments, blocking agents can be introduced at the same time that the sample is exposed to optical labels, e.g., as part of step 304. The blocking agents—due to their greater reactivity with the barcode sequences of oligonucleotides 104 of corresponding probes 100—preferentially hybridize to the corresponding probes, thereby preventing countersense barcode sequences of oligonucleotides 106 of optical labels 150 from doing so. In effect, the blocking agents out-compete the barcode sequences of oligonucleotides 106 of optical labels 150 for cross-reactivity binding sites.

In some embodiments, blocking agents can be introduced after exposure of the sample to optical labels in step 304. To the extent hybridization cross-reactivity occurs between the countersense barcode sequences of oligonucleotides 106 of optical labels and barcode sequences of oligonucleotides 104 of probes 100 in step 304, the blocking agents can assist with dehybridization (i.e., displacement) of cross-hybridized countersense barcode sequences, as they hybridize preferentially to the barcode sequences of oligonucleotides 104 of probes 100.

In some embodiments, to detect a particular target analyte A, the sample may only be contacted with blocking agents where the abundance of certain target analytes {B, C, D . . . } is high, and/or where the hybridization cross-reactivity of barcode sequence Sₐ is sufficiently high such that false detection represents a significant problem. As an example, consider a multiplexed sample analysis protocol involving labeling and visualization of biomarkers CD8, pan-cytokeratin, CD68, PD1, PDL-1, CD20, and FoxP3 in a tissue sample. When applying the probe for CD8, it may be advisable to contact the sample with a blocking agent for the oligonucleotide 104 sequence of probes that bind to pan-cytokeratin, because pan-cytokeratin is typically highly abundant in such samples.

In certain embodiments, for example, when a target analyte A of interest is present in the sample at a concentration M_{c}, and where the sample also contains another target analyte B bound to a probe D_{b}, a blocking agent can be introduced into the sample to block the oligonucleotide 104 barcode sequence of probe D_{b} when a concentration of target analyte B in the sample is, or estimated to be, 0.05 M_{c} or more (e.g., 0.10 M_{c} or more, 0.15 M_{c} or more, 0.20 M_{c} or more, 0.25 M_{c} or more, 0.30 M_{c} or more, 0.40 M_{c} or more, 0.50 M_{c} or more, 0.60 M_{c} or more, 0.70 M_{c} or more, 0.80 M_{c} or more, 1.00 M_{c} or more, 1.50 M_{c} or more, 2.00 M_{c} or more, 3.00 M_{c} or more, 5.00 M_{c} or more, 10.00 M_{c} or more, 20.00 M_{c} or more, 50.00 M_{c} or more, 100.00 M_{c} or more, or even more).

As another example, in certain embodiments, it may be advisable to contact the sample with a blocking agent for the oligonucleotide 104 sequence of a probe that binds to a marker such as CD20, because even though CD20 may be less abundant than pan-cytokeratin, the inherent hybridization cross-reactivity of the oligonucleotide of the optical label that associates with the CD8 probe may be relatively high for the oligonucleotide 104 sequence of the CD20 probe.

When a target analyte A of interest is present in the sample and is bound to a probe Da, where the sample also contains another target analyte B bound to a probe D_{b}, and where an optical label Rₐ is introduced that contains a countersense barcode sequence of oligonucleotide 106 that is nominally intended to selectively hybridize to the barcode sequence of probe Da but not to the barcode sequence of probe D_{b} for detection of target analyte A, the hybridization discrimination factor, H, can be defined as the ratio of the number of hybridizations of optical label Rₐ and probe Da, to the number of hybridizations of optical label Rₐ and probe D_{b}. When optical label Rₐ exhibits perfect specificity, H is infinite. However, when the specificity is less than perfect, the value of H is reduced.

In some embodiments, a blocking agent for the barcode sequence of oligonucleotide 104 of probe D_{b} can be introduced when the value of the hybridization discrimination factor between probes Da and D_{b} of target analytes A and B for optical label Rₐ is 1.0×10⁶ or less (e.g., 5.0×10⁵ or less, 1.0×10⁵ or less, 5.0×10⁴ or less, 1.0×10⁴ or less, 0.5×10³ or less, 1.0×10³ or less, 500 or less, 300 or less, 200 or less, 100 or less, 50 or less, 10 or less, 5 or less, 2 or less, or even less).

As a further example, in some embodiments, it may be advisable to contact the sample with a blocking agent for the oligonucleotide 104 sequence of a probe that binds to a marker such as PDL1, not because PDL1 is highly abundant nor (in this example) because the hybridization cross-reactivity between S′_{CD8} and S_{PDL1} is not high compared with the reactivity between S′_{CD8} and S_{CD8} (and other sequences), but because the assay demands an extremely low false-positive rate to be of practical benefit as a diagnostic test. For abundant target analytes, the sample may not be contacted with blocking agents, since the magnitude of false detection signals may be relatively small compared to the magnitude of detection signals corresponding to the abundant target analytes.

In some embodiments, for practical reasons, it can be advantageous to use as many different labeling reagents Rᵢ—each with a different (and unique) oligonucleotide 106 barcode sequence—as the total number of target analytes to be assayed in a particular protocol. It should be noted that the total number of target analytes in a particular protocol may be greater than the level of multiplexing (i.e., the number of different target analytes that are detected in a single cycle of the protocol). For example, for an assay that detects 20 different target analytes using 20 different optical labels, each of which contains a unique oligonucleotide 106 sequence, each individual target analytes can be interrogated without duplicating oligonucleotide 104 barcode sequences (or oligonucleotide 106 barcode sequences) in the protocol.

In some embodiments, a sample can be contacted with one or more blocking agents at an excess concentration relative to an optical label to ensure that hybridization cross-reactivity of the optical label is maintained at a suitably small level. For example, for a sample that is contacted with an optical label at a concentration of L_{c} in solution, the sample can be contacted with one or more blocking agents at a concentration of 2.0 L_{c} or more (e.g., 3.0 L_{c} or more, 4.0 L_{c} or more, 5.0 L_{c} or more, 7.0 L_{c} or more, 10.0 L_{c} or more, 15.0 L_{c} or more, 20.0 L_{c} or more, 50.0 L_{c} or more, 100 L_{c} or more, or even more).

In some embodiments, in an analysis protocol, the total number of target analytes that are assayed/detected can be 4 or more (e.g., 6 or more, 8 or more, 10 or more, 12 or more, 14 or more, 16 or more, 18 or more, 20 or more, 25 or more, 30 or more, 35 or more, 40 or more, 50 or more, 60 or more, 70 or more, 80 or more, 100 or more, 200 or more, 300 or more, 400 or more, 500 or more, 600 or more, 800 or more, 1000 or more, or even more). Further, in an analysis protocol, the number of different target analytes that are assayed/detected in a single detection cycle (i.e., the level of multiplexing) can be 1 or more (e.g., 2 or more, 3 or more, 4 or more, 5 or more, 6 or more, 7 or more, 8 or more, 10 or more, or even more).

In the foregoing examples, blocking agents include oligonucleotides with countersense barcode sequences {S′_{b}, S′_{c}, S′_{d} . . . } that preferentially hybridize to barcode sequences {S_{b}, S_{c}, S_{d} . . . }, which are associated with probes {D_{b}, D_{c}, D_{d} . . . } that bind to target species {B, C, D . . . }. As such, the blocking agents effectively block optical labels from hybridizing to the barcode sequences of the probes. It is also possible, however, to introduce blocking agents with barcode sequences {S_{b}, S_{c}, S_{d} . . . } that preferentially hybridize to countersense barcode sequences of oligonucleotides 106 of optical labels. These blocking agents effectively “inactivate” certain optical labels, preventing them from binding to any probes in the sample. Such methods can be useful, for example, when a sample is contacted with certain optical label for which a counterpart probes are not present in the sample (i.e., the target analytes corresponding to the optical labels are not present in the sample, and so no probes for the analytes are present in the sample). Such optical labels, if left unblocked, might exhibit some hybridization cross-reactivity with probes that are present in the sample, generating false positive signals. Such signals can be reduced or eliminated by blocking these optical labels.

By contacting the sample with any of the blocking agents described above, a large inventory of oligonucleotide 104 barcode sequences (and oligonucleotide 106 countersense barcode sequences) can be developed which are suitable for probes 100 that detect weakly expressed target analytes. Among the barcode sequences and countersense barcode sequences, it is not necessary that each oligonucleotide 106 countersense barcode sequence must have very low hybridization cross-reactivity for all but one oligonucleotide 104 barcode sequence. Instead, as described above, blocking agents can be used to effectively inactivate oligonucleotide 104 barcode sequences that are not of interest in a particular labeling and measurement cycle, ensuring that even if hybridization cross-reactivity would otherwise occur to a relevant (i.e., measureable) extent in the absence of such blocking agents, hybridization cross-reactivity is suppressed by the blocking agents. Reagents containing the barcode sequences and countersense barcode sequences, such as probes, optical labels, and blocking agents incorporating the barcode sequences and countersense barcode sequences, and kits containing these sequences/reagents (e.g., kits that include a set of sample labeling reagents), can be used for flexible and high-performance visualization of even weakly expressed target analytes during a multiplexed detection and visualization protocol.

The foregoing methods, reagents and reagent compositions, and kits can be used in any analysis protocol in which target analytes are interrogated individually, including multi-cycle analysis protocols in which a single target analyte is interrogated in each cycle of the protocol. The methods, reagents and reagent compositions, and kits can also be used in any analysis protocol in which target analytes are interrogated in small groups (e.g., 2, 3, 4, 5, 6, 8, 10 target analytes detected simultaneously) in one or more cycles of a the protocol.

When target analytes are interrogated in groups (e.g., in a cycle of an analysis protocol) by exposing probes in the sample to different types of optical labels at the same time, the sequences of oligonucleotides 104 of the probes and sequences of oligonucleotides 106 of the optical labels can be selected such that the hybridization cross-reactivity between each of the countersense barcode sequences of oligonucleotides 106 of a group of optical labels 150 for the group of target analytes and the barcode sequences of oligonucleotides 104 of the probes that bind to the group of target analytes is sufficiently low so that blocking agents may not need to be introduced to block barcode sequences for probes of the group of target analytes. However, blocking agents may still be introduced to block barcode sequences of probes bound to other target analytes in the sample that are not part of the group, inhibiting hybridization cross-reactivity between the countersense barcode sequences of oligonucleotides 106 of the optical labels for members of the group and the other target analytes that are not part of the group.

Similar considerations to those above regarding the use of blocking agents in protocols where optical labels 150 include an oligonucleotide 106 linked to an enzyme such as HRP also apply. Blocking agents can be used in both single cycle and multi-cycle protocols involving such optical labels, in protocols where target analytes are interrogated singly, and in protocols where target analytes are interrogated in groups (i.e., simultaneously). As noted above, when optical labels include an oligonucleotide linked to an enzyme, blocking agents for the oligonucleotide barcode sequences of all probe types except one can be introduced, or blocking agents for the oligonucleotide barcode sequences of some probe types but not all probe types other than one probe type corresponding to one target analyte of interest can be introduced.

Reagents and Conditions

In general, the various steps described herein can be implemented under a wide variety of conditions and with different reagents. Accordingly, the reagents and conditions described in this section should be understood to represent only examples of suitable reagents and conditions.

Typically, probes can be stored following preparation in a buffer solution that can include one or more of PBS, PBS-T, TBS, TBS-T, water, saline solution, and Kreb's buffer. The buffer solution can optionally include one or more blocking materials, such as (but not limited to) oligonucleotides.

Enzymes and other catalytic agents can also be stored following preparation in a buffer solution. The buffer can include one or more of PBS, PBS-T, TBS, TBS-T, water, saline solution, and Kreb's buffer. The buffer solution can be the same as, or different from, the buffer solution used to store the probes.

To promote hybridization between the probes and optical labels, the probes and optical labels can be immersed in a hybridization buffer. Suitable hybridization buffers can include DNA components, protein components, detergents, and/or chaotropic reagents at concentrations of between 5% and 20%.

To promote de-hybridization between the probes and optical labels, the probes and optical labels can be immersed in a de-hybridization buffer. Suitable de-hybridization buffers can include chaotropic reagents such as DMSO and/or formamide, at concentrations of between 60% and 90%.

To promote binding of a probe to a target analyte in a sample, the probe can be layered onto the sample in solution, e.g., by pipetting, and incubated with the sample. Following incubation, unbound probes can be washed from the sample using, for example, a buffer solution that includes one or more of PBS, PBS-T, TBS, TBS-T, water, saline solution, and Kreb's buffer.

The incubation time for any of the hybridization, reaction, binding, and de-hybridization steps described herein can be 10 minutes or more (e.g., 20 minutes or more, 30 minutes or more, 40 minutes or more, 60 minutes or more, 1 hour or more, 2 hours or more, 3 hours or more, 4 hours or more, 5 hours or more, 6 hours or more, 8 hours or more, 10 hours or more, 16 hours or more, 20 hours or more, 24 hours or more, 48 hours or more, 7 days or more, 30 days or more).

Compositions and Kits

The probes, optical labels, blocking agents, and other reagents, species, and moieties described herein can be included in a variety of kits featuring compositions that include the probes, optical labels, blocking agents, and other reagents, species, and moieties. In general, a kit is a package of one or more reagents, each of which is in the form of a composition. Compositions featuring any of the different probes, optical labels, blocking agents, and other reagents, species, and moieties described herein can be prepared and used for target analyte analysis as described herein. These compositions can be included in product kits, along with other features such as instructions for preparing compositions, and using the compositions for sample analysis. Product kits can be sealed or otherwise contained in a variety of different containers and housings.

In some embodiments, a composition—to which a sample can optionally be exposed—can include a plurality of probes. Each of the probes can include a capture moiety 102 and an oligonucleotide 104. The capture moieties 102 and oligonucleotides 104 can have any of the properties described herein. The probes can also optionally include any of the other features described herein.

Probes in a composition can be selected such that, when the compositions are used to label samples, different types of target analytes in the sample are labeled with different types of probes. Each type of probe can selectively bind to a different type of target analyte in the sample. Typically, probes of the same type each include the same oligonucleotide 104 so target analyte molecules of the same type are labeled with the same optical moiety. Probes of each type have a capture moiety that differs from the capture moieties of probes of other types, and typically (although not always) have an oligonucleotide 104 that differs from the oligonucleotides 104 of other types of probes.

Optical labels in a composition can be selected such that different types of optical labels selectively associate with (e.g., bind to) different types of probes. Typically, optical labels of the same type each include the same oligonucleotide 106 and so they associate with probes of one type, labeling the probes with the optical label. Optical labels of the same type generally include the same optical moiety 108, enzyme, or other species linked to oligonucleotide 106, so that the probes of the one type are coupled to the same optical moiety 108, enzyme, or other species linked to oligonucleotide 106. Optical labels of each type have an oligonucleotide 106 that typically (although not always) differs from the oligonucleotides 106 of other optical labels. In some embodiments, optical labels of each type have an optical moiety that differs from the optical moieties of the other optical labels.

In some embodiments, as described herein, a sample is exposed to optical labels in groups or pools, where each such group includes 2 or more (e.g., 3 or more, 4 or more, 5 or more, 6 or more, 8 or more, or even more) different optical labels. Within a composition, the optical labels can optionally be divided into sub-compositions, each of which contains a group or pool of optical labels. In certain embodiments, among sub-compositions, each optical label can be present in only one sub-composition. Alternatively, in some embodiments, one or more optical labels can be present in more than one sub-composition.

In some embodiments, optical labels are present in the same composition as the probes, blocking agents, and/or other components discussed above. In certain embodiments, optical labels are present in a different composition to which the sample is exposed after exposure to one or more compositions that include(s) the probes, blocking agents, and/or other components.

Blocking agents in a composition can be selected such that different types of blocking agents selectively associate with (e.g., bind to) different types of probes, and specifically, to oligonucleotides 104 of different types of probes. Typically, blocking agents of the same type each include the same oligonucleotide so probe oligonucleotides 104 of the same type are blocked by the same type of blocking agent.

In some embodiments, blocking agents are present in the same composition as the probes, optical labels, and/or other components discussed above. In certain embodiments, blocking agents are present in a different composition to which the sample is exposed after exposure to one or more compositions that include(s) the probes, optical labels, and/or other components.

Compositions can generally include probes, optical labels, blocking agents, and other reagents, species, and moieties and other components that are used to label a sample as described herein such that any number of different types of target analytes in the sample can be distinguishably labeled, visualized, identified, and quantified, in a spatially-resolved manner. In some embodiments, for example, compositions can be used to label 2 or more (e.g., 3 or more, 4 or more, 5 or more, 10 or more, 20 or more, 30 or more, 40 or more, 50 or more, 100 or more, 200 or more, 300 or more, 400 or more, 500 or more, 1000 or more, 2000 or more, 3000 or more, 4000 or more, 5000 or more, 10000 or more, or even more) different types of target analytes in the sample, and can contain distinguishable probes, optical labels, blocking agents, and other components of the same number, greater number, or lesser number.

Each population or group of probes, optical labels, blocking agents, or another component within a composition can generally include any number of probes, optical labels, blocking agents, and/or other component of a particular type. For example, the number of probes, optical labels, blocking agents, or another component of a particular type can be 1 or more (e.g., 2 or more, 3 or more, 5 or more, 10 or more, 50 or more, 100 or more, 200 or more, 300 or more, 500 or more, 1000 or more, 2000 or more, 3000 or more, 5000 or more, 7000 or more, 10000 or more, 30000 or more, 50000 or more, 100000 or more, 500000 or more, or even more).

In some embodiments, compositions can include one or more additional components. For example, in some embodiments, compositions can include one or more buffer solutions. Examples of suitable buffer solutions include, but are not limited to, saline-sodium citrate (SSC), phosphate-buffered saline (PBS), and tris-ethylenediaminetetraacetic acid (tris-EDTA).

One or more of the compositions described above can be included as part of a kit for analyzing a sample. The kit can include a housing or packaging that encloses the contents of the kit. Compositions can be contained within containers in the kit; such containers can be formed from a wide variety of materials, including (but not limited to) plastics and glass.

Kits can optionally include a variety of other components as well. For example, in some embodiments, kits can include one or more dehybridization reagents. Examples of such reagents include, but are not limited to, sodium hydroxide, dimethyl sulfoxide (DMSO), formamide, SDS, methanol, and ethanol.

In certain embodiments, kits can include one or more buffer solutions. Examples of suitable buffer solutions include, but are not limited to, saline-sodium citrate (SSC), phosphate-buffered saline (PBS), and tris-ethylenediaminetetraacetic acid (tris-EDTA).

Kits can also optionally include instructions printed or otherwise recorded on any of a variety of different media (e.g., paper, computer readable storage media) that describe the use of certain components of the kits in assays targeting RNAs of various types in samples.

Imaging Systems and Components

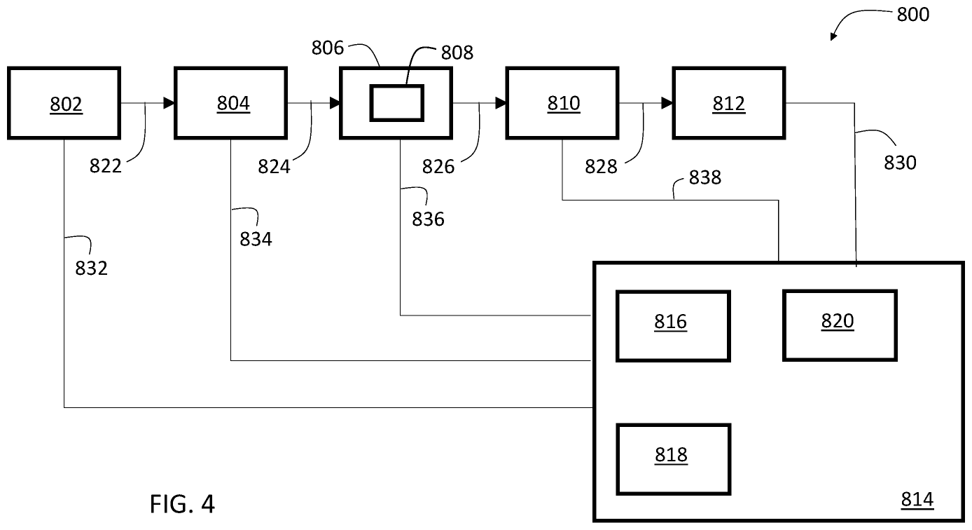

FIG. 4 is a schematic diagram showing a system 800 for acquiring multiple spectrally resolved images of a sample. System 800 can measure light emitted from, transmitted from, and/or reflected by a sample that includes one or more of the probes and corresponding optical labels described herein. The measured light generally includes contributions from each of the optical labels (e.g., the optical moieties) present in the sample, and system 800 can analyze the multispectral image information encoded in the measured light, decomposing the image information to isolate contributions to the measured light from each of the optical labels in the sample. The decomposition yields, for each optical label in the sample, a set of amplitude or intensity measurements as a function of position within the sample. The amplitude or intensity measurements can be used to quantify the amount of each optical label, and therefore the amount of each target analyte, at each position in the sample.

A light source 802 provides light 822 to light conditioning optics 804. Light 822 can be incoherent light, such as light generated from a filament source for example, or light 822 can be coherent light, such as light generated by a laser. Light 822 can be either continuous-wave (CW) or time-gated (i.e., pulsed) light. Further, light 822 can be provided in a selected portion of the electromagnetic spectrum. For example, light 822 can have a central wavelength and/or a distribution of wavelengths that falls within the ultraviolet, visible, infrared, or other regions of the spectrum.

Light conditioning optics 804 can be configured to transform light 822 in a number of ways. For example, light conditioning optics 804 can spectrally filter light 822 to provide output light in a selected wavelength region of the spectrum. Alternatively, or in addition, light conditioning optics can adjust the spatial distribution of light 822 and the temporal properties of light 822. Incident light 824 is generated from light 822 by the action of the elements of light conditioning optics 804.

Incident light 824 is directed to be incident on sample 608 mounted on illumination stage 806. Stage 806 can provide means to secure sample 808, such as mounting clips or other fastening devices. Alternatively, stage 806 can include a movable track or belt on which a plurality of samples 808 are affixed. A driver mechanism can be configured to move the track in order to successively translate the plurality of samples, one at a time, through an illumination region on stage 806, whereon incident light 824 impinges. Stage 806 can further include translation axes and mechanisms for translating sample 808 relative to a fixed position of illumination stage 806. The translation mechanisms can be manually operated (e.g., threaded rods) or can be automatically movable via electrical actuation (e.g., motorized drivers, piezoelectric actuators).

In response to incident light 824, emitted light 826 emerges from sample 808. Emitted light 826 can be generated in a number of ways. For example, in some embodiments, emitted light 826 corresponds to a portion of incident light 824 transmitted through sample 808. In other embodiments, emitted light 826 corresponds to a portion of incident light 824 reflected from sample 808. In yet further embodiments, incident light 824 can be absorbed by sample 808, and emitted light 826 corresponds to fluorescence emission from sample 808 (e.g., from fluorescent components in sample 808) in response to incident light 824. In still further embodiments, sample 808 can be luminescent, and may produce emitted light 826 even in the absence of incident light 824. In some embodiments, emitted light 826 can include light produced via two or more of the foregoing mechanisms.

Light collecting optics 810 are positioned to received emitted light 826 from sample 808. Light collecting optics 810 can be configured to collimate emitted light 826 when light 826 is divergent, for example. Light collecting optics 810 can also be configured to spectrally filter emitted light 826. Filtering operations can be useful, for example, in order to isolate a portion of emitted light 826 arising via one of the mechanisms discussed above from light arising via other processes. For example, the methods described herein are used to determine accurate estimates of the fluorescence spectra of one or more labeling moieties in a sample. Light collecting optics 810 can be configured to filter out non-fluorescence components of emitted light 826 (e.g., components corresponding to transmitted and/or reflected incident light). Further, light collecting optics 810 can be configured to modify the spatial and/or temporal properties of emitted light 826 for particular purposes in embodiments. Light collecting optics 810 transform emitted light 826 into output light 828 which is incident on detector 812.

Detector 812 includes one or more elements such as CCD sensors and/or CMOS sensors configured to detect output light 828. In some embodiments, detector 812 can be configured to measure the spatial and/or temporal and/or spectral properties of light 828. Detector 812 generates an electrical signal that corresponds to output light 828, and is communicated via electrical communication line 830 to electronic control system 814.

Electronic control system 814 includes a processor 816, a display device 818, and a user interface 820. In addition to receiving signals corresponding to output light 828 detected by detector 812, control system 814 sends electrical signals to detector 812 to adjust various properties of detector 812. For example, if detector 812 includes a CCD sensor, control system 814 can send electrical signals to detector 812 to control the exposure time, active area, gain settings, and other properties of the CCD sensor.

Electronic control system 814 also communicates with light source 802, light conditioning optics 804, illumination stage 806, and light collecting optics 810 via electrical communication lines 832, 834, 836, and 838, respectively. Control system 814 provides electrical signals to each of these elements of system 800 to adjust various properties of the elements. For example, electrical signals provided to light source 802 can be used to adjust the intensity, wavelength, repetition rate, or other properties of light 822. Signals provided to light conditioning optics 804 and light collecting optics 810 can include signals for configuring properties of devices that adjust the spatial properties of light (e.g., spatial light modulators) and for configuring spectral filtering devices, for example. Signals provided to illumination stage 806 can provide for positioning of sample 808 relative to stage 806 and/or for moving samples into position for illumination on stage 806, for example.

Control system 814 includes a user interface 820 for displaying system properties and parameters, and for displaying captured images of sample 808. User interface 820 is provided in order to facilitate operator interaction with, and control over, system 800. Processor 816 includes a storage device for storing image data captured using detector 812, and also includes computer software that embodies instructions to processor 816 that cause processor 816 to carry out control functions, such as those discussed above for example. Further, the software instructions cause processor 816 to mathematically manipulate the images captured by detector 812 and to carry out the steps of decomposing images obtained by system 800 into contributions from particular optical labels in the sample.

In some embodiments, light conditioning optics 804 include an adjustable spectral filter element such as a filter wheel or a liquid crystal spectral filter. The filter element can be configured to provide for illumination of the sample using different light wavelength bands. Light source 802 can provide light 822 having a broad distribution of spectral wavelength components. A selected region of this broad wavelength distribution is allowed to pass as incident light 824 by the filter element in light conditioning optics 804, and directed to be incident on sample 808. Subsequently, the wavelength of the filter pass-band in light conditioning optics 804 is changed to provide incident light 824 having a different wavelength. Spectrally-resolved images can also be recorded by employing a light source 802 having multiple source elements generating light of different wavelengths, and alternately turning the different source elements on and off to provide incident light 684 having different wavelengths.

Light collecting optics 810 can include configurable spectral filter elements similar to those discussed above in connection with light conditioning optics 804. Therefore, spectral resolution can be provided on the excitation side of sample 808 (e.g., via light conditioning optics 804) and on the emission side of sample 808 (e.g., via light collecting optics 810).

The result of collecting multiple, spectrally resolved images of a sample is an “image stack” where each image in the stack is a two-dimensional image of the sample corresponding to a particular wavelength band or set of wavelength bands. Conceptually, the set of images can be visualized as forming a three-dimensional matrix, where two of the matrix dimensions are the spatial length and width of each of the images, and the third matrix dimension is the spectral index. The spectral index may directly correlate with wavelength (i.e., the spectral index value may be a wavelength value, such as a central wavelength, for a corresponding measurement wavelength band) or more generally, the spectral index may be an identifier that represents a more complex combination of wavelength bands in which an optical signal is measured.

The set of spectrally resolved images can be referred to as a “spectral cube” of images. As used herein, a “pixel” in such a set of images (or image stack or spectral cube), refers to a common spatial location for each of the images. Accordingly, a pixel in a set of images includes a value associated with each image at the spatial location corresponding to the pixel.

To isolate contributions from each of multiple optical labels in a sample to the image information contained in a multispectral image stack, spectral unmixing methods can be used. Spectral unmixing is a technique that quantitatively separates contributions in an image that arise from spectrally different sources. For example, a sample may contain three different spectral sources. The three different spectral sources may each have different absorption spectra. Typically, the individual absorption spectra of the spectral sources are known before they are used, or they can be measured. Images of the sample under illumination will contain, in the most general case, spectral contributions from each of the three spectral sources.

Spectral unmixing decomposes one or more images that include contributions from multiple spectral sources into a set of component images (the “unmixed images”) that correspond to contributions from each of the spectral sources within the sample. Thus, if the sample includes three different spectral sources (e.g., three different labeling agents and/or optical labels), then an image of the sample can be separated into three unmixed images, each unmixed image reflecting contributions principally from only one of the spectral sources.

The unmixing procedure essentially corresponds to decomposing an image into a set of spectral eigenstates. In many embodiments, the eigenstates are known beforehand, as discussed above. In other embodiments, the eigenstates can sometimes be determined using techniques such as principal component analysis. In either case, once the eigenstates have been identified, an image can be decomposed by calculating a set of values, usually as a coefficient matrix, that corresponds to the relative weighting of each of the eigenstates in the overall image. The contributions of each of the individual eigenstates can then be separated out to yield the unmixed image set.

Aspects of spectral unmixing are described in U.S. Pat. Nos. 10,126,242 and 7,555,155, and in PCT Patent Publication No. WO2005/040769, the entire contents of each of which are incorporated herein by reference.

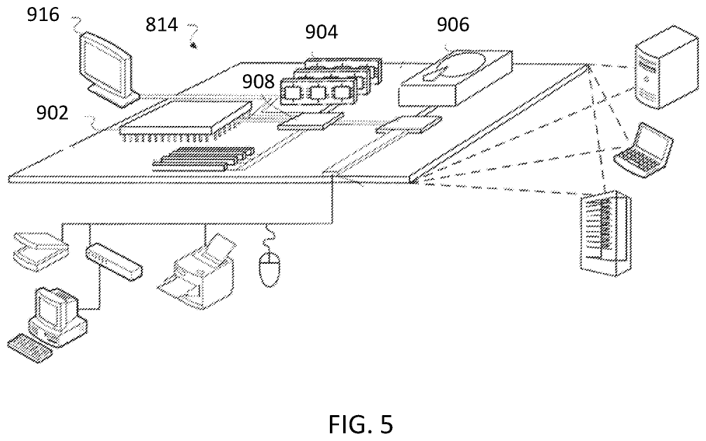

FIG. 5 shows an example of an electronic control system 814, which may be used with the systems, methods, compositions, and kits disclosed herein. Electronic control system can include one or more processors 902 (e.g., corresponding to processor 816 in FIG. 4), memory 904, a storage device 906 and interfaces 908 for interconnection. The processor 902 can process instructions for execution within the electronic control system 814, including instructions stored in the memory 904 or on the storage device 906. For example, the instructions can instruct the processor 902 to perform any of the analysis and control steps disclosed herein.

The memory 904 can store executable instructions for processor 902, information about parameters of the system such as excitation and detection wavelengths, and measured spectral image information. The storage device 906 can be a computer-readable medium, such as a floppy disk device, a hard disk device, an optical disk device, or a tape device, a flash memory or other similar solid state memory device, or an array of devices, including devices in a storage area network or other configurations. The storage device 906 can store instructions that can be executed by processor 902 described above, and any of the other information that can be stored by memory 904.

In some embodiments, electronic control system 814 can include a graphics processing unit to display graphical information (e.g., using a GUI or text interface) on an external input/output device, such as display 916. The graphical information can be displayed by a display device (e.g., a CRT (cathode ray tube) or LCD (liquid crystal display) monitor) for displaying any of the information, such as measured and calculated spectra and images, disclosed herein. A user can use input devices (e.g., keyboard, pointing device, touch screen, speech recognition device) to provide input to the electronic control system 814.

The methods disclosed herein can be implemented by electronic control system 814 (and processors 902 and 816) by executing instructions in one or more computer programs that are executable and/or interpretable on the electronic control system 814. These computer programs (also known as programs, software, software applications or code) include machine instructions for a programmable processor, and can be implemented in a high-level procedural and/or object-oriented programming language, and/or in assembly/machine language. For example, computer programs can contain the instructions that can be stored in memory 904, in storage unit 906, and/or on a tangible, computer-readable medium, and executed by processor 902 (processor 816) as described above. As used herein, the term “computer-readable medium” refers to any computer program product, apparatus and/or device (e.g., magnetic discs, optical disks, memory, Programmable Logic Devices (PLDs), ASICs, and electronic circuitry) used to provide machine instructions and/or data to a programmable processor, including a machine-readable medium that receives machine instructions.

Generally, electronic control system 814 can be implemented in a computing system to implement the operations described above. For example, the computing system can include a back end component (e.g., as a data server), or a middleware component (e.g., an application server), or a front end component (e.g., a client computer having a graphical user-interface), or any combination thereof.

### OTHER EMBODIMENTS

While this disclosure describes specific implementations, these should not be construed as limitations on the scope of the disclosure, but rather as descriptions of features in certain embodiments. Features that are described in the context of separate embodiments can also generally be implemented in combination in a single embodiment. Conversely, various features that are described in the context of a single embodiment can also be implemented in multiple embodiments separately or in any suitable sub-combination. Moreover, although features may be described above as present in certain combinations and even initially claimed as such, one or more features from a claimed combination can generally be excised from the combination, and the claimed combination may be directed to a sub-combination or variation of a sub-combination.

In addition to the embodiments expressly disclosed herein, it will be understood that various modifications to the embodiments described may be made without departing from the spirit and scope of the disclosure. Accordingly, other embodiments are within the scope of the following claims.

What is claimed is:

1. A method for detecting multiple target analytes in a biological sample, the method comprising:
(a) contacting a biological sample with a plurality of different types of probes, wherein each different type of probe comprises a capture moiety that selectively binds to a different target analyte in the sample, and an oligonucleotide having a sequence that is unique among other types of probes in the plurality of different types of probes;
(b) binding an optical label to one of the different types of probes, wherein the optical label comprises an optical moiety linked to a labeling oligonucleotide, and wherein the labeling oligonucleotide comprises a sequence that hybridizes to the oligonucleotide sequence of the one type of probe;
(c) contacting the sample with a composition comprising at least one blocking agent, wherein the at least one blocking agent comprises an oligonucleotide having a sequence that hybridizes to the oligonucleotide of another type of probe from among the different types of probes; and
(d) obtaining an image of the sample comprising information corresponding to one or more locations of the one type of probe in the sample.

2. The method of claim 1, further comprising contacting the sample with the composition prior to binding the optical label to the one of the different types of probes.

3. The method of claim 1, further comprising contacting the sample with the composition during the binding the optical label to the one of the different types of probes.

4. The method of claim 1, further comprising contacting the sample with the composition after binding the optical label to the one of the different types of probes.

5. The method of claim 1, wherein the capture moiety comprises at least one of an antibody and an antibody fragment.

6. The method of claim 1, wherein the capture moiety comprises an aptamer.

7. The method of claim 1, wherein the capture moiety comprises at least one of a protein and a peptide.

8. The method of claim 1, wherein the capture moiety comprises a ribonucleic acid.

9. The method of claim 1, wherein binding the optical label to the one of the different types of probes comprises hybridizing the optical label to the one of the different types of probes.

10. The method of claim 1, wherein the at least one blocking agent does not comprise an optical moiety or an enzyme.

11. The method of claim 1, wherein the optical moiety comprises a fluorescent dye species.

12. The method of claim 1, wherein the optical moiety comprises an enzyme.

13. The method of claim 12, further comprising, prior to obtaining the image of the sample, exposing the sample to a second composition comprising a tyramide-conjugated optical moiety to deposit the optical moiety in the sample in proximity to the one of the different types of probes.

14. The method of claim 1, wherein the composition comprises multiple different types of blocking agents, and wherein each different type of blocking agent comprises an oligonucleotide having a sequence that hybridizes to the oligonucleotide of a different one of the other types of probes.

15. The method of claim 1, further comprising:
removing the optical label from the sample; and
repeating steps (b) and (c) with at least one additional optical label.

16. The method of claim 1, wherein the sample comprises n different types of target analytes, and wherein the composition comprises (n−1) different types of blocking agents.

17. The method of claim 1, wherein the composition comprises blocking agents comprising oligonucleotides with sequences that are complementary to oligonucleotides of one or more other types of probes from among the plurality of different types of probes.

18. The method of claim 1, wherein the image comprises fluorescence emission information for the sample.

19. The method of claim 1, wherein the plurality of different types of probes comprises at least 20 different types of probes.

20. The method of claim 1, wherein the oligonucleotide of the probe comprises at least 20 nucleotides.

21. The method of claim 1, wherein a concentration of the optical label in the sample is M_{c}, and a concentration of a blocking agent in the sample is 1.5 M_{c} or more.

22. The method of claim 1, wherein a value of a hybridization discrimination factor between two different types of probes and the optical label is 100 or less.

23. A kit, comprising:
a first composition comprising a plurality of different types of probes, wherein each different type of probe comprises a capture moiety that selectively binds to a different target analyte, and an oligonucleotide having a sequence that is unique among other types of probes in the plurality of different types of probes;
an optical label comprising an optical moiety linked to a labeling oligonucleotide, wherein the labeling oligonucleotide comprises a sequence that is at least partially complementary to the oligonucleotide sequence of one of the different types of detection molecules; and
a second composition comprising at least one blocking agent, wherein the at least one blocking agent comprises an oligonucleotide having a sequence that is at least partially complementary to the oligonucleotide sequence of another type of probe from among the different types of probes.

24. The kit of claim 23, wherein the capture moiety comprises at least one of an antibody, an antibody fragment, an aptamer, a protein, and a peptide.

25. The kit of claim 23, wherein the capture moiety comprises a ribonucleic acid.

26. The kit of claim 23, wherein the at least one blocking agent does not comprise an optical moiety or an enzyme.

27. The kit of claim 23, wherein the optical moiety comprises a fluorescent dye species.

28. The kit of claim 23, wherein the optical moiety comprises an enzyme.

29. The kit of claim 23, wherein:
the first composition comprises n different types of target analytes;
the second composition comprises a plurality of sub-compositions;
each sub-composition comprises less than n different types of blocking agents; and
each sub-composition is housed within a separate container.

30. The kit of claim 23, wherein the second composition comprises multiple different types of blocking agents, and wherein each type of blocking agent comprises an oligonucleotide having a sequence that is at least partially complementary to the oligonucleotide sequence of a different type of probe from among the different types of probes.
